(19) United States
(12) Patent Application Publication
(10) Pub. No.: US 2019/0046964 A1
(43) Pub. Date: Feb. 14, 2019

(54) SYNTHESIS OF SAPO-34 AND USE IN CHLOROMETHANE TO OLEFINS REACTIONS

(71) Applicant: SABIC Global Technologies B.V., Bergen op Zoom (NL)

(72) Inventors: Dustin Fickel, Sugar Land, TX (US); Neeta Kulkarni, Sugar Land, TX (US); Kaiwalya Sabnis, Sugar Land, TX (US)

(21) Appl. No.: 16/076,739

(22) PCT Filed: Dec. 21, 2016
(86) PCT No.: PCT/IB2016/057890

§ 371 Date: Aug. 9, 2018

Related U.S. Application Data
(60) Provisional application No. 62/309,117, filed on Mar. 16, 2016.

(51) Int. Cl. B01J 29/85; C07C 1/26; C07C 11/02; B01J 35/02; B01J 37/04; B01J 37/08

(52) U.S. Cl. CPC B01J 29/85; C07C 1/26; C07C 11/02; C07C 2529/85; B01J 37/04; B01J 37/08; B01J 35/023

(57) ABSTRACT

Disclosed are methods of producing SAPO-34 and MeAPSO-34 molecular sieves having nano-crystal morphology and optionally a hierarchical structure. Also disclosed are methods and systems of using said molecular sieve for catalyzing the reaction of alkyl halides to light olefins.

### CROSS REFERENCE TO RELATED APPLICATIONS

This application claims the benefit of priority of U.S. Provisional Patent Application No. 62/309,117, filed Mar. 16, 2016, which is hereby incorporated by reference in its entirety.

### BACKGROUND OF THE INVENTION

### A. Field of the Invention

The invention generally concerns a process of making silicoaluminophosphate (SAPO) and metal incorporated silicoaluminophosphate (MeAPSO) molecular sieve catalysts that can be used to catalyze the reaction of alkyl halides to light olefins. The process can result in catalysts having nano-sized crystal morphology, hierarchical structure of micropores and mesopores, or a combination of both nano-sized morphology and hierarchical structure.

### B. Description of Related Art

Light olefins such as ethylene and propylene are used by the petrochemical industry to produce a variety of key chemicals that are then used to make numerous downstream products. By way of example, both of these olefins are used to make a multitude of plastic products that are incorporated into many articles of manufacture. FIGS. 1A and 1B provide examples of products generated from ethylene (FIG. 1A) and propylene (FIG. 1B).

Methane activation to higher hydrocarbons, especially to light olefins, has been the subject of great interest over many decades. Recently, the conversion of methane to light olefins via a two-step process that includes conversion of methane to methyl halide, particularly to methyl mono-halide, for example, to methyl chloride, followed by conversion of the halide to light olefins as well as the direct methanol to olefin (MTO) reaction has attracted great attention. Microporous zeolite (e.g., ZSM-5) or zeolite type catalysts (e.g., SAPO-34) have been commonly employed for these methyl chloride (or other methyl halide) and methanol conversion reactions. However, the selectivity to a desired olefin (e.g., propylene), rapid catalyst deactivation due to carbon deposition (coking), and the synthesis cost of the catalyst remain the major challenges for scale-up and commercial success of the reaction. Coking can occur as zeolite pores are blocked by formed methylbenzene species trapped inside the cages of the framework. The continual filling of zeolite cages with methylbenzene species (and eventual naphthalene and larger polyaromatic species) leads to a diffusion limitation into and out of the zeolite framework.

SAPO catalysts have an open microporous structure with regularly sized channels, pores or “cages.” These materials are sometimes referred to as “molecular sieves” in that they have the ability to sort molecules or ions based primarily on the size of the molecules or ions. SAPO materials are both microporous and crystalline and have a three-dimensional crystal framework of PO₄⁺, AlO₄⁻ and SiO₄ tetrahedra. SAPO-34 and MeAPSO-34 catalysts have been prepared by a range of methods. Many of the conventional preparations of SAPO or MeAPSO materials include hydrothermal synthesis where an aqueous solution of alumina precursor, silica precursor, phosphorous precursor and templating agent are heated under pressure to form a crystalline product. (See, for example, Razavian et al. in “Recent Advances in Silicoaluminophosphate Nanocatalysts Synthesis Techniques and Their Effects on Particle Size Distribution, Reviews on Advancement of Material Science, 2011, Vol. 29, pp. 83-99). Other additive such as mesopore forming agents or crystal growth inhibitors can be added to the aqueous solution to produce larger pores and/or inhibit crystal growth. One of the issues with this conventional process is that the size of the resulting crystal particles can be too large. This can lead to the aforementioned diffusion issues with reactants and products and ultimately reduced catalytic performance.

Another conventional route concerns the production of “dry gels” or “xerogels” of alumina, phosphorous oxide, and silica. These dry Si/Al/P gels are dried and subsequently reconstituted in a solution that includes a templating agent, and, optionally other additives. (See, for example, Chinese patent publication CN101993093B to Zhongmin et al., Hui et al. in China Petroleum Processing and Petrochemical Technology 2012, Vol. 14, No. 3, pp 68-74 and Chinese patent publication CN103420388A to Wang et al). The addition of a templating agent after the drying step also results in larger crystal particles and reduced catalytic performance due, in part, to the aforementioned diffusion issues.

While there are many methods to make SAPO catalysts or MeAPSO catalysts, in particular SAPO-34 catalysts or MeAPSO-34 catalysts, these catalysts still suffer in that they are prone to deactivation at high pressures and temperatures when used in alkyl halide to light olefin reaction processes. The deactivation (e.g., coking) can be due, in part, to reduced diffusion kinetics of reactants and products through the catalytic materials.

### SUMMARY OF THE INVENTION

A discovery has been made that provides a solution to the aforementioned issues concerning silicoaluminophosphate (SAPO) and metal incorporated silicoaluminophosphate (MeAPSO) molecular sieve catalysts used in alkyl halide to light olefin (e.g., C₂-C₄ olefins) reaction processes. The discovery is premised on a process of producing SAPO-34 and MeAPSO-34 (M-SAPO-34) catalysts having either a nano-crystal morphology, a hierarchical structure with both mesopores (pores having a diameter of 2 nm to 50 nm) and micropores (pores having a diameter of up to 2 nm), or a combination of both. In particular, the process includes drying an aqueous solution that includes water, a silicon source, an aluminum source, a phosphorous source, a templating agent, and, optionally, a metal source, to obtain a dried SAPO-34 or MeAPSO-34 precursor material. The dried precursor material can be completely, or substantially devoid, of water (e.g., less than 10 wt. %, less than 5 wt. %, less than 2 wt. %, less than 1 wt. % water). This dried precursor material can be subsequently contacted with water to form a suspension or slurry. The water can be in liquid form (“water assisted dry synthesis”) or water vapor or steam form (“water vapor/steam assisted dry synthesis”). The produced suspension or slurry can be subjected to crystallization conditions to obtain crystal particles. The crystal particles can then be calcined to produce the nano-sized catalysts of the present invention. Without wishing to be bound by theory, it is believed that the nano-sized morphology can be obtained through the drying and reconstitution steps. Drying the aqueous solution removes water and excess templating agent that has not been occluded in the zeolite growth nuclei. Subsequent reconstitution of the dried precursor material in water to form the suspension or slurry results in a situation where the added water is theoretically more dilute in terms zeolite growth nuclei, which lessens the probability of two nuclei agglomerating to form a larger crystal. Also, the lack of free templating agent in the added water further reduces the kinetics of crystal growth.

Additionally, the produced crystal particles can be further reduced in size by adding a crystal growth modifier (e.g., long chain hydrocarbons or polyglycols such as polyethylene glycol (PEG), cetyltrimethylammonium bromide (CTAB), polyimines, polyethyleneimine (PEI), etc.) to the initial aqueous solution comprising water, a silicon source, an aluminum source, a phosphorous source, and a templating agent. It is believed that the crystal growth modifier can attach to the exterior surface of the zeolite nuclei in the aqueous solution. Upon drying the solution to produce the dried precursor material, the crystal growth modifier can have a steric hindrance affect by reducing the likelihood of two nuclei coming in contact with or combining with one another. When the dried precursor material is subsequently reconstituted in water, then these nuclei are more likely to crystallize in isolation due to the aforementioned steric hindrance characteristics provided by the crystal growth modifier. Additionally, there can be instances where the crystal growth modifier can become encapsulated in a crystal particle (e.g., where two or more nuclei overcome the steric hindrance effects and combine together and continue to grow). In such instances, mesopores can be formed by removal of the encapsulated crystal growth modifier via calcination. Therefore, crystal growth modifiers can have a dual purpose by limiting the size of the resulting SAPO or MeAPSO crystals through steric hindrance and by introducing mesopores into the crystals. The end result can be crystal particles having a nano-crystal morphology and a hierarchical structure with both mesopores and micropores.

A mesopore forming agent can also be added to the initial aqueous solution that includes water, a silicon source, an aluminum source, a phosphorous source, a templating agent, and optionally a crystal growth modifier. A difference between mesopore forming agents and crystal growth modifiers is that the mesopore forming agents are designed to become incorporated into growing crystal nuclei (e.g., carbon nanotubes). Therefore, the addition of mesopore forming agents into the initial aqueous solution allows for the production of a hierarchical structure with both mesopores and micropores when these agents are ultimately removed in the calcination step.

The SAPO or MeAPSO crystals produced by the processes of the present invention have faster diffusion kinetics of reactants and products through the crystal, thereby providing improved conversion and selectivity in the alky halide to olefin reaction and higher activity. Further, the nano-sized crystal particles of the present have more surface area when compared with the larger conventionally produced catalysts, which further increases the efficiencies of the catalyst of the present invention.

In one embodiment of the present invention, there is disclosed a method for preparing a silicoaluminophosphate (SAPO)-34 molecular sieve. The method can include: (a) obtaining an aqueous mixture that can include water, a silicon source, an aluminum source, a phosphorous source, and a templating agent; (b) drying the mixture to obtain a dried material that includes a SAPO-34 precursor material loaded with the templating agent; (c) contacting the dried material with water and subjecting the material to crystallization conditions to obtain a SAPO-34 crystalline material loaded with the templating agent; and (d) removing the templating agent from the crystalline material to obtain the SAPO-34 molecular sieve. Contacting the dried material with liquid water can occur prior to the crystallization step. Contacting the dried material with water vapor or steam can occur during the crystallization step. In one aspect of the method, step (c) includes suspending the dried material in an aqueous solution to form a suspension and subjecting the suspension to a temperature of 180° C. to 210° C. for a desired amount of time (e.g., about 12 hours to 36 hours) under autogenous pressure to obtain the SAPO-34 crystalline material loaded with the templating agent. In another aspect of the method, step (c) includes contacting the dried material with water vapor or steam and subjecting the material to a temperature of 180° C. to 210° C. for a desired amount of time (e.g., about 12 hours to 36 hours) under autogenous pressure to obtain the SAPO-34 crystalline material loaded with the templating agent. The SAPO-34 molecular sieve obtained by these methods can have a microporous structure and can be in particulate form having an average particle size of 50 nm to 500 nm or 50 nm to 200 nm. In one aspect of the method, the aqueous mixture in step (a) has a molar composition of:

aR:bSiO₂:cAl₂O₃:dP₂O₅:eH₂O
where R is the templating agent, 0<a≤4, 0<b≤1, 0<c≤1, 0<d≤1, and e is 30 to 80.

In another embodiment of the present invention, the aqueous mixture in step (a) of the method can further include a crystal growth modifier, a mesopore-forming agent, or both. The dried material from step (b) and the crystalline material from step (c) can each be loaded with the templating agent, the crystal growth modifier and/or the mesopore-forming agent. The templating agent, the crystal growth modifier and/or the mesopore-forming agent can each be removed from the crystalline material to obtain the SAPO-34 molecular sieve. A SAPO-34 molecular sieve obtained by this method has a hierarchical structure of micropores and mesopores and is in particulate form having an average particle size of 50 nm to 500 nm, or 20 nm to 500 nm. In one aspect, the aqueous mixture in step (a) has a molar composition of:

aR:bSiO₂:cAl₂O₃:dP₂O₅:eH₂O:fX,
where: R is the templating agent; X is the crystal growth modifier, a mesopore-forming agent or both; 0<a≤4; 0<b≤1; 0<c≤1; 0<d≤1; e is 30 to 80; and 0<f≤1.

The crystal growth modifier can be polyethylene glycol (PEG), cetyltrimethylammonium bromide (CTAB), a polyimine, polyethyleneimine (PEI) or any combination thereof. In some embodiments, crystal growth modifiers are capable of forming mesopores in the zeolite structure. Other mesopore forming agents can include carbon nanotubes. In another instance, the aqueous mixture further includes a metal source. The metal in the metal source can be manganese, magnesium, copper, cobalt, iron, nickel, germanium, or zinc. The metal (Me) source can be a metal oxide. In this instance, the SAPO-34 molecular sieve obtained from the method is a metal incorporated (Me)ASPO-34 molecular sieve with a metal incorporated into the SAPO-34 framework and the aqueous mixture in step (a) can have a molar composition of:

aR:bSiO₂:cAl₂O₃:dP₂O₅:eH₂O:gMe_{y}O_{z},
where: R is the templating agent; 0<a≤4; 0<b≤1; 0<c≤1; 0<d≤1; e is 30 to 80; 0<g≤1; y is 1 to 2; and z is 1 to 3.

In another aspect, the aqueous mixture in step (a) has a molar composition of:

aR:bSiO₂:cAl₂O₃:dP₂O₅:eH₂O:fX:gMe_{y}O_{z},
where: R is the templating agent; X is the crystal growth modifier, the mesopore-forming agent or both; 0<a≤4, 0<b≤1; 0<c≤1; 0<d≤1; e is 30 to 80; 0<f≤1, and g is 0<g≤1; y is 1 to 2; and z is 1 to 3.
   The silicon source used in any of the current methods can be colloidal silica, fumed silica, or tetraethyl orthosilicate, the aluminum source can be aluminum isopropoxide, and the phosphorous source can be phosphoric acid. The templating agent can be an amine, a quaternary ammonium salt or both. In a particular aspect, the amine is diethylamine, triethylamine, or morpholine, and the quaternary ammonium salt is tetraethylammonium hydroxide. In one aspect, the drying step (b) removes substantially all of the templating agent from the aqueous mixture other than the templating agent that is loaded into the SAPO-34 precursor material. In another aspect, the drying step (b) includes subjecting the mixture to a temperature of 80° C. to 110° C. for a desired period of times (e.g., 12 to 48 hours); and the removing step (d) includes subjecting the crystalline material to a temperature of 500° C. to 600° C. for 3 hours to 10 hours.

In further embodiments, a silicoaluminophosphate (SAPO)-34 or MeAPSO-34 molecular sieve is provided that is obtained by any of the methods disclosed in the present invention. In one aspect, the SAPO-34 or MeAPSO-34 molecular sieve has a hierarchical structure of micropores and mesopores and/or an average particle size of 50 nm to 500 nm, preferably a particle size of 50 nm to 200 nm. Particle size can be determined using Scanning Transmission Microscopy (SEM).

In still another embodiment of the present invention, there is disclosed a system for producing olefins. The system can include an inlet for a feed including the alkyl halide discussed above and throughout this specification, a reaction zone that is configured to be in fluid communication with the inlet, and an outlet configured to be in fluid communication with the reaction zone to remove an olefin hydrocarbon product from the reaction zone. The reaction zone can include the SAPO-34 or MeAPSO molecular sieves of the present invention, the feed and the olefin hydrocarbon product. In some instances, the olefin hydrocarbon product can include ethylene, propylene, or both. The system of the current embodiments can further include a collection device that is capable of collecting the olefin hydrocarbon product.

Also disclosed, are methods for converting an alkyl halide to an olefin. A method can include contacting a SAPO-34 or MeAPSO-34 molecular sieve catalyst of the present invention with a feed that includes an alkyl halide under reaction conditions sufficient to produce an olefin hydrocarbon product that includes C₂-C₄ olefins. The alkyl halide used in the method can be a methyl halide (e.g., methyl chloride, methyl bromide, methyl fluoride, or methyl iodide, or any combination thereof). In certain aspects of the method, the reaction for converting an alkyl halide to an olefin occurs in a fixed bed process or reactor, fluid catalytic cracking (FCC) process or reactor or fluidized circulating bed process or reactor. Reaction conditions for the conversion to an alkyl halide can include a temperature from 300° C. to 500° C., a pressure of 5 atm or less, and a weighted hourly space velocity (WHSV) of 0.5 to 10 h⁻¹, preferably a temperature of 450° C., a pressure of 1 atm, and a WHSV of 3 h⁻¹. The method can also involve collecting or storing the produced olefin hydrocarbon product and using the produced olefin hydrocarbon product to produce a petrochemical or a polymer.

In the context of the present invention 33 embodiments are disclosed. Embodiment 1 is a method for preparing a silicoaluminophosphate (SAPO)-34 molecular sieve is described that includes; (a) obtaining an aqueous mixture that can include water, a silicon source, an aluminum source, a phosphorous source, and a templating agent; (b) drying the mixture to obtain a dried material that includes a SAPO-34 precursor material loaded with the templating agent; (c) contacting the dried material with water and subjecting the material to crystallization conditions to obtain a SAPO-34 crystalline material loaded with the templating agent; and (d) removing the templating agent from the crystalline material to obtain the SAPO-34 molecular sieve. Embodiment 2 is the method of embodiment 1, wherein step (c) includes suspending the dried material in an aqueous solution to form a suspension and subjecting the suspension to a temperature of 180° C. to 210° C. for 12 hours to 36 hours under autogenous pressure to obtain the SAPO-34 crystalline material loaded with the templating agent. Embodiment 3 is the method of embodiment 1, wherein step (c) includes contacting the dried material with water vapor or steam and subjecting the material to a temperature of 180° C. to 210° C. for 12 hours to 36 hours under autogenous pressure to obtain the SAPO-34 crystalline material loaded with the templating agent. Embodiment 4 is the method of any one of embodiments 1 to 3, wherein the obtained SAPO-34 molecular sieve has a microporous structure and is in particulate form having an average particle size of 50 nm to 500 nm or 50 nm to 200 nm. Embodiment 5 is the method of any one of embodiments 1 to 4, wherein the aqueous mixture in step (a) has a molar composition of:

aR:bSiO₂:cAl₂O₃:dP₂O₅:eH₂O

wherein R is the templating agent, and a is 0<a≤4, b is 0<b≤, c is 0<c≤1, d is 0<d≤1, and e is 30 to 80. Embodiment 6 is the method of any one of embodiments 1 to 3, wherein the aqueous mixture in step (a) further includes a crystal growth modifier, a mesopore-forming agent or both; the dried material from step (b) and the crystalline material from step (c) are each loaded with the templating agent and the crystal growth modifier, the mesopore-forming agent, or both; and the templating agent and the crystal growth modifier and/or the mesopore-forming agent are each removed from the crystalline material to obtain the SAPO-34 molecular sieve. Embodiment 7 is the method of embodiment 5, wherein the obtained SAPO-34 molecular sieve has a hierarchical structure of micropores and mesopores and is in particulate form having an average particle size of 50 nm to 500 nm, or 20 nm to 500 nm. Embodiment 8 is the method of any one of embodiments 6 to 7, wherein the aqueous mixture in step (a) has a molar composition of:

aR:bSiO₂:cAl₂O₃:dP₂O₅:eH₂O:fX,

wherein R is the templating agent and X is the crystal growth modifier, the mesopore-forming agent or both, and a is 0<a≤4, b is 0<b≤1, c is 0<c≤1, d is 0<d≤1, e is 30 to 80, and f is 0<f≤1. Embodiment 9 is the method of any one of embodiments 6 to 8, wherein mesopor-forming agent is a polyethylene glycol (PEG), cetyltrimethylammonium bromide (CTAB), a polyimine, preferably, or a polyethyleneimine (PEI), carbon nanotubes, or any combination thereof. Embodiment 10 is the method of claim 9, wherein the crystal growth modifier is a polyethylene glycol (PEG), a cetyltrimethylammonium bromide (CTAB), a polyimine, preferably, polyethyleneimine (PEI). Embodiment 11 is the method of any one of embodiments 1 to 8, wherein the aqueous mixture further includes a metal (Me) source, wherein Me is manganese, magnesium, copper, cobalt, iron, nickel, germanium, or zinc. Embodiment 12 is the method of embodiment 11, wherein the Me source is a metal oxide. Embodiment 13 is the method of any one of claims 10 to 11, wherein the obtained the SAPO-34 molecular sieve is a MeASPO-34 molecular sieve with Me incorporated into the SAPO-34 framework. Embodiment 14 is the method of any one of embodiments 11 to 13, wherein the aqueous mixture in step (a) has a molar composition of:

aR:bSiO₂:cAl₂O₃:dP₂O₅:eH₂O:gMe_{y}O_{z},

wherein R is the templating agent, and a is 0<a≤4, b is 0<b≤1, c is 0<c≤1, d is 0<d≤1, e is 30 to 80, and g is 0<g≤1, wherein y is 1 to 2 and z is 1 to 3. Embodiment 15 is the method of any one of embodiments 11 to 13, wherein the aqueous mixture in step (a) has a molar composition of:

aR:bSiO₂:cAl₂O₃:dP₂O₅:eH₂O:fX:gMe_{y}O_{z},

wherein R is the templating agent and X is the crystal growth modifier, the mesopore-forming agent or both, and a is 0<a≤4, b is 0<b≤1, c is 0<c≤1, d is 0<d≤1, e is 30 to 80, f is 0<f≤1, and g is 0<g≤1, wherein y is 1 to 2 and z is 1 to 3. Embodiment 16 is the method of any one of embodiments 1 to 15, wherein the silicon source is colloidal silica, fumed silica, or tetraethyl orthosilicate, the aluminum source is aluminum isopropoxide, and the phosphorous source is phosphoric acid. Embodiment 17 is the method of any one of embodiments 1 to 16, wherein the templating agent is an amine, a quaternary ammonium salt or both. Embodiment 18 is the method of embodiment 17, wherein the amine is diethylamine, triethylamine, or morpholine, and the quaternary ammonium salt is tetraethylammonium hydroxide. Embodiment 19 is the method of any one of embodiments 1 to 18, wherein the drying step (b) removes substantially all of the templating agent from the aqueous mixture other than the templating agent that is loaded into the SAPO-34 precursor material. Embodiment 20 is the method of any one of embodiments 1 to 19, wherein the drying step (b) includes subjecting the mixture to a temperature of 80° C. to 110° C.; and the removing step (d) includes subjecting the crystalline material to a temperature of 500° C. to 600° C. for 3 hours to 10 hours. Embodiment 21 is a silicoaluminophosphate (SAPO)-34 molecular sieve obtained by the method of any one of embodiments 1 to 20.

Embodiment 22 is a silicoaluminophosphate (SAPO)-34 or MeAPSO-34 molecular sieve having an average particle size of 50 nm to 500 nm, preferably a particle size of 50 nm to 200 nm, and, optionally, a hierarchical structure of micropores and mesopores. Embodiment 23 is the silicoaluminophosphate (SAPO)-34 molecular sieve of embodiment 22, that further includes a metal (Me) incorporated into its framework structure.

Embodiment 24 is a system for producing olefins. The system includes an inlet for a feed that includes an alkyl halide; a reaction zone that is configured to be in fluid communication with the inlet, wherein the reaction zone includes the silicoaluminophosphate (SAPO)-34 molecular sieve of any one of embodiments 22 to 23; and an outlet configured to be in fluid communication with the reaction zone to remove an olefin hydrocarbon product from the reaction zone. Embodiment 25 is the system of embodiment 24, wherein the reaction zone further includes the feed and the olefin hydrocarbon product. Embodiment 26 is the system of embodiment 25, wherein the olefin hydrocarbon product includes ethylene and propylene. Embodiment 27 is the system of any one of embodiments 24 to 26, further including a collection device that is capable of collecting the olefin hydrocarbon product.

Embodiment 28 is a method for converting an alkyl halide to an olefin. The method includes contacting the silicoaluminophosphate (SAPO)-34 molecular sieve of any one of embodiments 22 to 23 with a feed that includes an alkyl halide under reaction conditions sufficient to produce an olefin hydrocarbon product that includes C₂-C₄ olefins. Embodiment 29 is the method embodiment 28, wherein the alkyl halide is a methyl halide, preferably methyl chloride, methyl bromide, methyl fluoride, or methyl iodide, or any combination thereof. Embodiment 30 is the method of any one of embodiments 28 to 29, wherein the reaction occurs in a fixed bed reactor, a fluid catalytic cracking (FCC) reactor, or fluidized circulating bed reactor. Embodiment 31 is the method of any one of embodiments 28 to 30, wherein the reaction conditions include a temperature from 300° C. to 500° C., a pressure of 5 atm or less, and a weighted hourly space velocity (WHSV) of 0.5 to 10 h⁻¹. Embodiment 32 is the method of any one of embodiments 23 to 31 that further includes collecting or storing the produced olefin hydrocarbon product. Embodiment 33 is the method of any one of embodiments 23 to 32 that further includes using the produced olefin hydrocarbon product to produce a petrochemical or a polymer.

The following includes definitions of various terms and phrases used throughout this specification.

The term “mesopore” is a pore in a material with diameters between 2 nm and 50 nm. Porous materials can be classified into several kinds depending on their size. According to IUPAC notation, microporous materials have pore diameters of less than 2 nm and macroporous materials have pore diameters of greater than 50 nm; the mesoporous category thus falls in between micropore and macropore. For example, a mesoporous material can have pore diameters of 2 to 50 nm. Pore diameters can be determined using Barrett-Joyner Halenda (BJH) method.

The following includes definitions of various terms and phrases used throughout this specification.

The term “catalyst” means a substance which alters the rate of a chemical reaction. “Catalytic” means having the properties of a catalyst.

The term “conversion” means the mole fraction (i.e., percent) of a reactant converted to a product or products.

The term “selectivity” refers to the percent of converted reactant that went to a specified product, for example C₂-C₄ olefin selectivity is the % of alkyl halide that formed C₂-C₄ olefins.

The term “template” or “templating agent” means any synthetic and/or natural material that provides at least one nucleation site where ions can nucleate and grow to form crystalline material.

The term “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %”, “vol. %”, or “mol. %” refers to a weight, volume, or molar percentage of a component, respectively, based on the total weight, the total volume of material, or total moles, that includes the component. In a non-limiting example, 10 grams of component in 100 grams of the material is 10 wt. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The catalysts of the present invention can “comprise,” “consist essentially of,” or “consist of” particular ingredients, components, compositions, etc. disclosed throughout the specification. With respect to the transitional phase “consisting essentially of,” in one non-limiting aspect, a basic and novel characteristic of the catalysts of the present invention are their ability to selectivity produce light olefins, and in particular, ethylene and propylene, from alkyl halides (e.g., methyl chloride).

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description.

### BRIEF DESCRIPTION OF THE DRAWINGS

FIGS. 1A and 1B depict illustrations of various chemicals and products that can be produced from ethylene (FIG. 1A) and propylene (FIG. 1B).

FIG. 2 depicts a system for producing olefins from alkyl halides.

FIG. 3 shows scanning electron microscopy (SEM) images of the SAPO-34 molecular sieve of the present invention at 82.2 kx magnification.

FIG. 4 shows graphs of chloromethane conversion versus time on stream for a SAPO-34 catalyst and a mesopore SAPO-34 catalyst of the present invention, a comparative SAPO-34 catalyst, and a comparative mesopore SAPO-34 catalyst.

FIG. 5 shows graphs of chloromethane conversion vs. time on stream for a MnAPSO-34 catalyst and a mesopore MnAPSO-34 catalyst of the present invention, a comparative SAPO-34 catalyst, and a comparative MnAPSO-34 catalyst.

FIG. 6 shows graphs of the percentage of chloromethane conversion and ethylene and propylene selectivities versus time on stream in hours for MnAPSO-34 catalyst of the present invention and a conventionally prepared MnAPSO-34 catalyst.

### DETAILED DESCRIPTION OF THE INVENTION

A discovery has been made that provides a solution to the deactivation of SAPO-34 or MeAPSO-34 catalysts used in alkyl halide to olefin reactions. The discovery is premised on the ability to produce SAPO-34 and MeAPSO-34 (M-SAPO-34) nano-sized catalysts that can optionally have a hierarchical mesoporous and microporous structure. The method of the present invention provides an elegant way to produce these features, which can result in more efficient catalytic performance when compared with catalysts prepared by conventional synthesis routes. Without wishing to be bound by theory, it is believed that the nano-sized crystals provide a high surface area for catalytic activity. The pore structure (e.g., micropores and/or a hierarchical pore structure) increases the diffusion kinetics of reactants and products. Thus, the catalysts of the present invention are less prone to deactivation and also maintain high catalytic activity when compared with catalysts produced by conventional synthesis routes. Without wishing to be bound by theory, the control of crystal size and/or the introduction of a hierarchical structure have a two-fold consequence. By preparing smaller crystals using the method of the present invention, the mean free path length can be reduced allowing increased diffusion and faster turnover from reactants to products. The hierarchical structure, if present, has increased mesopores that can enable reactants and products to escape the zeolite framework more easily.

These and other non-limiting aspects of the present invention are discussed in further detail in the following sections with reference to the Figures.

### A. Methods of Making SAPO Catalysts

1. Nano-Crystal SAPO-34 and MeAPSO-34 Synthesis

The SAPO zeolites (e.g., SAPO-34 and MeAPSO-34 zeolites) can be prepared using a gel containing aluminum (Al), phosphorus (P) and silicon (Si) compounds, and optionally a metal source, with structure-directing agents under crystallization conditions in a template reaction. The addition of a structure-directing or template agent/ion effects the pre-organization provided by the coordination sphere and can result in modification of physical/chemical/electronic properties of the template complex formed. Methods of making the SAPO-34 catalysts are exemplified in the Examples section and in the discussion below. Step 1 of the method can include obtaining an aqueous mixture that includes water, a silicon source, an aluminum source, a phosphorous source, and a templating agent. The silicon source, aluminum source, phosphorous source and templating agent are discussed in further detail in the Materials section below. In some embodiments, the silicon, aluminum and phosphorous source are oxides and the synthesis mixture can have a molar composition of:

aR:bSiO₂:cAl₂O₃:dP₂O₅:eH₂O,

where R is the templating agent, and 0<a≤4, 0<b≤1, 0<c≤1, 0<d≤1, and e is 30 to 80, and a, b, c, d, e are the molar amounts of templating agent, silica, aluminum, phosphorous oxide and water.

Non-limiting nano-crystal SAPO-34 catalysts can include a molar composition where a is 0.8, b is 0.65, c is 1, d is 1, and e is 40-50; a is 0.9, b is 0.65, c is 1, d is 1, and e is 40-50; a is 1, b is 0.65, c is 1, d is 1, and e is 40-50; a is 1.05, b is 0.65, c is 1, d is 1, and e is 40-50; a is 1.5, b is 0.65, c is 1, d is 1, and e is 40-50; a is 2, b is 0.65, c is 1, d is 1, and e is 40-50; a is 2.5, b is 0.65, c is 1, d is 1, and e is 40-50; a is 3, b is 0.65, c is 1, d is 1, and e is 40-50; a is 3.5, b is 0.65, c is 1, d is 1, and e is 40-50; a is 4, b is 0.65, c is 1, d is 1, and e is 40-50; a is 2, b is 0.4, c is 1, d is 1, and e is 60; a is 2, b is 0.6, c is 1, d is 1, and e is 60; a is 2, b is 0.8, c is 1, d is 1, and e is 60.

The synthesis mixture of a MeASPO zeolite can have the molar composition of:

aR:bSiO₂:cAl₂O₃:dP₂O₅:eH₂O:gMe_{y}O_{z},
where R is the templating agent, Me is the metal, and 0<a≤4, b is 0<b≤1, c is 0<c≤1, d is 0<d≤1, e is 30 to 80, and g is 0<g≤1, where y is 1 to 2 and z is 1 to 3.
   Non-limiting nano-crystal MeAPSO-34 catalysts can include a molar composition where a is 0.8, b is 0.65, c is 1, d is 1, e is 40-50, and g is 0.05; a is 1, b is 0.65, c is 1, d is 1, e is 40-50, and g is 1; a is 0.8, b is 0.55, c is 1, d is 1, e is 40-50, and g is 0.2; a is 2, b is 0.4, c is 1, d is 1, and e is 60, and g is 0.05; a is 2, b is 0.4, c is 1, d is 1, and e is 60, and g is 0.1; a is 2, b is 0.4, c is 1, d is 1, and e is 60, and g is 0.15; a is 2, b is 0.4, c is 1, d is 1, and e is 60, and g is 0.2; a is 2, b is 0.6, c is 1, d is 1, and e is 60, and g is 0.05; a is 2, b is 0.6, c is 1, d is 1, and e is 60, and g is 0.1; a is 2, b is 0.6, c is 1, d is 1, e is 60, and g is 0.15; a is 2, b is 0.6, c is 1, d is 1, e is 60, and g is 0.2; a is 2, b is 0.8, c is 1, d is 1, e is 60, and g is 0.05; a is 2, b is 0.8, c is 1, d is 1, e is 60, and g is 0.1; a is 2, b is 0.8, c is 1, d is 1, e is 60, and g is 0.15.

The synthesis mixture can be mixed together using known zeolite mixing techniques at 20° C. to 30° C. at standard pressures until a homogeneous gel is obtained (e.g., for about 1 hour to 24 hours). The gel includes at least 30 to 80 moles of water. Without wishing to be bound by theory, it is believed that during the mixing supersaturation of the inorganic reagents is reached and the zeolite/SAPO nuclei begin to condense in the solution. During the process of forming the nuclei, it is also believed that the organic templates are occluded into the nuclei. Mixing can be continued and the gel can be aged for a period of time at room temperature and pressure. For example, the gel can be aged for 0 to 24 hours at 20 to 60° C. at standard pressure. In some embodiments, mixing can be discontinued and the gel can be aged.

In step 2 of the method, the synthesis material can be heated at standard pressures to remove most of the water and non-occluded template from the gel to obtain a dried gel. This is in contrast to conventional methods to produce SAPO type materials, which heat the gel without drying under pressure to crystallize the SAPO materials (e.g., hydrothermal process). In a non-limiting example, the gel can be dried at 85° C. to 95° C., or 90° C. until a dried material is obtained (e.g., for 12 to 36 hours). The dried material can have a gel form or a particulate form. The dried material can have an amorphous type structure. The dried material can have a water content of less than 5 wt. %, 2 wt. %, 1 wt. % based on the total weight of the dried material, or be substantially devoid of water. Water content can be determined by thermogravimetric (TGA) analysis (for determination of weight loss) in combination with a mass spectrometer (for detection and identification of the gases (e.g., water) released from the sample when heated). The dried material includes a SAPO-34 precursor material loaded with the templating agent. Without wishing to be bound by theory, it is believed that after drying the gel, the only remaining template in the dried material is that which is occluded in the pores of the zeolite nuclei during their formation.

In step 3 of the method, the dried material can be subjected to crystallization conditions suitable to produce a SAPO (e.g., SAPO-34 or MeAPSO-34) crystalline material loaded with templating agent. The dried material can be contacted with sufficient water, water vapor or steam at temperatures and pressures suitable to wet and/or disperse the dried material and subsequently effect crystallization. Crystal growth can be performed in a pressure vessel, such as an autoclave using autogenous pressure, by a temperature-difference method, temperature-reduction method, or a metastable-phase technique. In a particular embodiment, the crystal growth is performed in an autoclave. In some embodiments, the dried material can be suspended in an aqueous solution to form a suspension. This suspension can be heated under autogenous pressure for a desired amount of time to obtain the SAPO (e.g., SAPO-34 or MeAPSO-34) crystalline material loaded with the templating agent (e.g., templating agent occluded in the pores of the crystalline material). In certain embodiments, the dried material can be contacted with water vapor or steam at a temperature of heated under autogenous pressure for a desired amount of time to obtain the SAPO crystalline material loaded with the templating agent. In such a method, the dried material and liquid water never come in contact with each other. Average crystallization temperatures can range from 180° C. to 210° C., and all temperatures therebetween including 181° C., 182° C., 183° C., 184° C., 185° C., 186° C., 187° C., 188° C., 189° C., 190° C., 191° C., 192° C., 193° C., 194° C., 195° C., 196° C., 197° C., 198° C., or 199° C., 200° C., 201° C., 202° C., 203° C., 204° C., 205° C., 206° C., 207° C., 208° C., or 209° C. Heating can be performed for 12 hours to 50 hours and all periods of time there between including 15 hours, 20 hours, 25 hours, 30 hours, 35 hours, 40 hours, 45 hours, 50 hours. Without wishing to be bound by theory, it is believed that since there is little to no free template available in the dispersion, the growth of the zeolite/SAPO crystals is arrested, which results in the formation of small nano-crystals. The nano-crystalline material loaded with template can be removed from the vessel and isolated using known methods (e.g., vacuum or gravity filtration, centrifugation, or the like).

In step 4, the nano-crystalline material loaded with template can be heated in the presence of an oxygen source (e.g., calcined in a flow of air) to remove the templating agent from the crystalline material with a microporous structure. In a particular instance, a SAPO-34 or a MeAPSO-34 molecular sieve is formed.

### 2. Synthesis of SAPO and MeAPSO with a Crystal Growth Modifier and/or Mesopore Forming Agent

In one aspect, a crystal growth modifier and/or a mesopore-forming agent is used to produce SAPO and MeAPSO materials. In step 1 of either of the above SAPO or MeAPSO methods, the aqueous synthesis mixture can include a crystal growth modifier agent and/or mesopore-forming agent. As discussed above, the crystal growth modifier can attach to the exterior surface of the zeolite nuclei in solution in step 1. In step 2, when the solution is dried, the crystal growth modifier can act as a barrier between two crystals and inhibits combination of the crystals. In step 3, when the dry gel is dispersed in water then the dried material (e.g., nuclei) are more likely to crystallize in isolation due to the steric hindrance, thereby resulting in small particles.

When a mesopore-forming agent is used, SAPO and MeAPSO materials with a hierarchical pore structure are produced. The hierarchical SAPO nano-crystal can include both micropores, as well as, mesopores integrated into their crystal structure. These hierarchical catalysts are formed with mesopore-forming agents. In some embodiments, the crystal growth modifier can be a mesopore-forming agent.

The synthesis mixture for a SAPO-34 nano-crystal with a crystal growth modifier and/or a mesopore-forming agent can have molar composition of:

aR:bSiO₂:cAl₂O₃:dP₂O₅:eH₂O:fX,
where R is the templating agent and X is the crystal growth modifier, the mesopore-forming agent or both, and 0<a≤4, 0<b≤1, 0<c≤1, 0<d≤1, e is 30 to 80, and 0<f≤1.

If both the mesopores-forming agent and the crystal growth modifier are used, f is the total molar amount. In some embodiments, a 0 wt. % to 10 wt. % of crystal growth modifier can be used. In certain embodiments, a 0 wt. % to 10 wt. % of mesopore-forming agent can be used. The nano-crystal SAPO-34 catalysts can include a molar ratios previously described for SAPO-34 catalysts of the present invention in combination with the molar ratios of the crystal growth modifier and/or the mesopore-forming agent. In some embodiments, f ranges from 0.01 to 0.06, 0.01, 0.02, 0.04, 0.06, 0.08, 0.09, 1, or any range or value there between. Non-limiting examples of molar compositions for the nano-crystal SAPO-34 catalysts with a crystal growth modifier are: a is 2, b is 0.4, c is 1, d is 1, e is 60, and f is 0.01, a is 2, b is 0.4, c is 1, d is 1, e is 60, and f is 0.02, a is 2, b is 0.4, c is 1, d is 1, e is 60, and f is 0.03, a is 2, b is 0.4, c is 1, d is 1, e is 60, and f is 0.05. Non-limiting examples of molar compositions for the nano-crystal SAPO-34 catalysts with a mesopore forming agent are: a is 2, b is 0.4, c is 1, d is 1, e is 60, and f is 0.01, a is 2, b is 0.4, c is 1, d is 1, e is 60, and f is 0.02, a is 2, b is 0.4, c is 1, d is 1, e is 60, and f is 0.03, a is 2, b is 0.4, c is 1, d is 1, e is 60, and f is 0.05. Non-limiting examples of molar compositions for the nano-crystal SAPO-34 catalysts with a crystal growth modifier and a mesopore forming agent are: a is 2, b is 0.4, c is 1, d is 1, e is 60, and f is 0.01, a is 2, b is 0.4, c is 1, d is 1, e is 60, and f is 0.02, a is 2, b is 0.4, c is 1, d is 1, e is 60, and f is 0.03, a is 2, b is 0.4, c is 1, d is 1, e is 60, and f is 0.05, a is 2, b is 0.4, c is 1, d is 1, e is 60, and f is 0.2, a is 2, b is 0.4, c is 1, d is 1, e is 60, and f is 0.3, a is 2, b is 0.4, c is 1, d is 1, e is 60, and f is 0.4, a is 2, b is 0.4, c is 1, d is 1, e is 60, and f is 0.5.

The synthesis mixture MeAPSO-34 nano-crystal can have a molar composition of:

aR:bSiO₂:cAl₂O₃:dP₂O₅:eH₂O:fX:gMe_{y}O_{z},
where R is the templating agent and X is the crystal growth modifier, the mesopore-forming agent or both and 0<a≤4, 0<b≤1, 0<c≤1, 0<d≤1, 30 to 80, 0<f≤1, and 0<g≤1, y is 1 to 2, and z is 1 to 3.
   If both the mesopores-forming agent and the crystal growth modifier are used, f is the total molar amount. The nano-crystal MeAPSO-34 catalysts can include a molar ratios previously described for MeAPSO-34 catalysts of the present invention in combination with the molar ratios of the crystal growth modifier and/or the mesopore-forming agent. In some embodiments, f ranges from 0.01 to 0.06, 0.01, 0.02, 0.04, 0.06, 0.08, 0.09, 1 or any range or value there between. Non-limiting examples of molar compositions for the nano-crystal MeAPSO-34 catalysts where a is 2, b is 0.4, c is 1, d is 1, e is 60, f is 0.01, and g is 0.05; a is 2, b is 0.6, c is 1, d is 1, e is 60, f is 0.01, and g is 0.05; a is 2, b is 0.4, c is 1, d is 1, e is 60, f is 0.01, and g is 0.1; a is 2, b is 0.4, c is 1, d is 1, e is 60, f is 0.02, and g is 0.05; a is 2, b is 0.4, c is 1, d is 1, e is 60, f is 0.02, and g is 0.1; a is 2, b is 0.4, c is 1, d is 1, e is 60, f is 0.03, and g is 0.05; a is 2, b is 0.4, c is 1, d is 1, e is 60, f is 0.03, and g is 0.1, a is 2, b is 0.4, c is 1, d is 1, e is 60, f is 0.02, and g is 0.05; a is 2, b is 0.4, c is 1, d is 1, e is 60, f is 0.02, and g is 0.1; a is 2, b is 0.4, c is 1, d is 1, e is 60, f is 0.03, and g is 0.05; a is 2, b is 0.4, c is 1, d is 1, e is 60, f is 0.03, and g is 0.1; a is 2, b is 0.4, c is 1, d is 1, e is 60, f is 0.04, and g is 0.05; a is 2, b is 0.4, c is 1, d is 1, e is 60, f is 0.04, and g is 0.1.

These synthesis mixtures can be crystallized to form nano-crystal SAPO materials using the Steps 1-4 above and the procedures in the Examples. When mesopore-forming agents are used increased numbers of mesopores are realized, where the larger mesopore-forming agent had integrated itself into the SAPO crystal during the crystallization process. Having a hierarchical structure (e.g., micropores and mesopores structure) can allow more efficient diffusion of molecules into and out of the SAPO micropores, which assist in inhibiting deactivation of the catalyst in the alkyl halides to olefins reaction. When mesopore-forming agents and crystal growth modifiers are used, nano-crystals with increased numbers of mesopores are realized, where the larger mesopore-forming agent had integrated itself into the SAPO crystal during the crystallization process.

### B. Nano-Crystal SAPO-34 and MeAPSO-34 Catalysts

The SAPO-34 or MeAPSO-34 nano-crystal produced using the methods above and in the Examples above can have any type of morphology. Non-limiting examples of morphologies include a wire, a particle, a sphere, a rod, a needle, a tetrapod, a hyper-branched structure, a tube, a cube, a plate, or mixtures thereof. The crystalline SAPO catalysts can have an average particle size of 50 nm to 500 nm or 50 nm to 200 nm. The average particle size of 50 nm to 200 nm includes all average particle sizes between 50 nm to 200 nm, for instance 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, and all values in between. Particle size can be determined using Scanning Electron Spectroscopy (SEM).

In some embodiments, the SAPO catalyst can include micropores. An average pore diameter of the microporous SAPO zeolite of the present invention can range from 0.01 nm to 2 nm, or 0.02 nm, 0.03 nm, 0.04 nm, 0.05 nm, 0.06 nm, 0.07 nm, 0.08 nm, 0.09 nm, 1 nm, 1.1 nm, 1.2 nm, 1.5 nm, 2 nm or any range or value there between. In some embodiments, the SAPO catalyst can include micropores and mesopores. An average pore diameter of the hierarchical SAPO zeolite of the present invention can have 100% of micropores in a range from 0.01 nm to 2 nm, or 0.01 nm, 0.02 nm, 0.03 nm, 0.04 nm, 0.05 nm, 0.06 nm, 0.07 nm, 0.08 nm, 0.09 nm, 1.0 nm or any range or value therebetween and 0 to 10% mesopores in a range of can have mesopores in a range from 2 nm to 50 nm, or 2 nm, 3 nm, 4 nm, 5 nm, 6 nm, 7 nm, 8 nm, 9 nm, 10 nm, 20 nm, 30 nm, 40 nm 50 nm or any range or value there between. A pore volume of SAPO-34 and the MeAPSO-34 materials can range from 0.2 cc/g to 0.4 cc/g, or 0.22 cc/g, 0.23 cc/g, 0.24 cc/g, 0.25 cc/g, 0.26 cc/g, 0.27 cc/g, 0.29 cc/g, 0.30 cc/g, 0.31 cc/g, 0.32 cc/g, 0.33 cc/g, 0.34 cc/g, 0.35 cc/g 0.36 cc/g, 0.37 cc/g, 0.38 cc/g, 0.39 cc/g, 0.40 cc/g or any range or value therebetween. Pore diameter and pore volume can be determined using micropore analyzer, nitrogen absorption, BJH method for mesopores, and Horvath-Kawazoe technique for micropores.

### C. Olefin Production

1. Methods and Systems

The nano- and/or hierarchical crystal SAPO-34 and MeAPSO-34 catalysts of the present invention help to catalyze the conversion of alkyl halides to C₂-C₄ olefins such as ethylene, propylene and butenes.

Conditions sufficient for olefin production (e.g., ethylene, propylene and butylene) include temperature, time, alkyl halide concentration, space velocity, and pressure. The temperature range for olefin production may range from about 300° C. to 500° C., preferably ranging 350° C. to 450° C. A weight hourly space velocity (WHSV) of alkyl halide higher than 0.5 h⁻¹ can be used, preferably between 1.0 and 10 h⁻¹, more preferably between 2.0 and 3.5 h⁻¹. The conversion of alkyl halide can be carried out at a pressure less than 145 psig (1 MPa) and preferably less than 73 psig (0.5 MPa), or at atmospheric pressure. The conditions for olefin production may be varied based on the type of the reactor.

The reaction of the methods and system disclosed herein can occur in a fixed bed process or reactor, fluid catalytic cracking (FCC)-type process or reactor or a circulating catalyst bed process or reactor. It is also envisioned the method and systems may also include the ability to regenerate used/deactivated catalyst in a continuous process such as in a fluid catalytic cracking (FCC)-type reactor or a circulating catalyst bed reactor. The method and system can further include collecting or storing the produced olefin hydrocarbon product along with using the produced olefin hydrocarbon product to produce a petrochemical or a polymer.

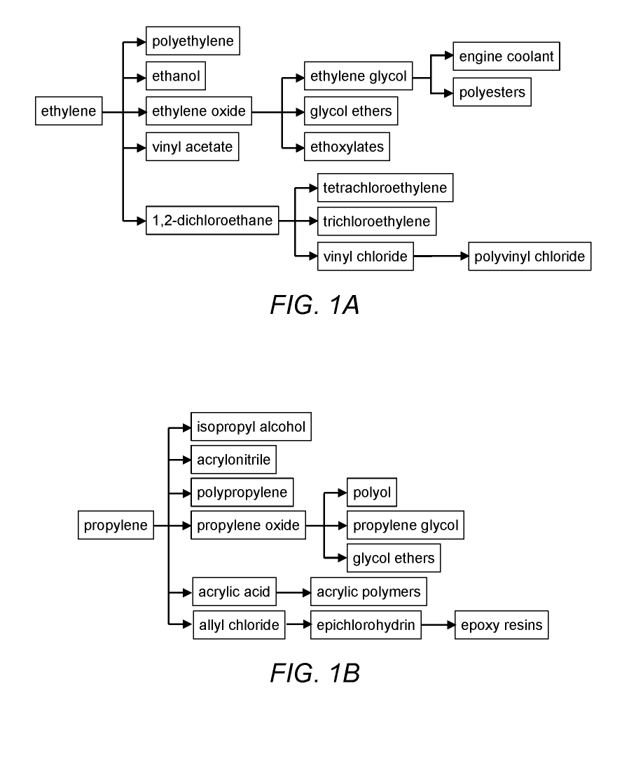

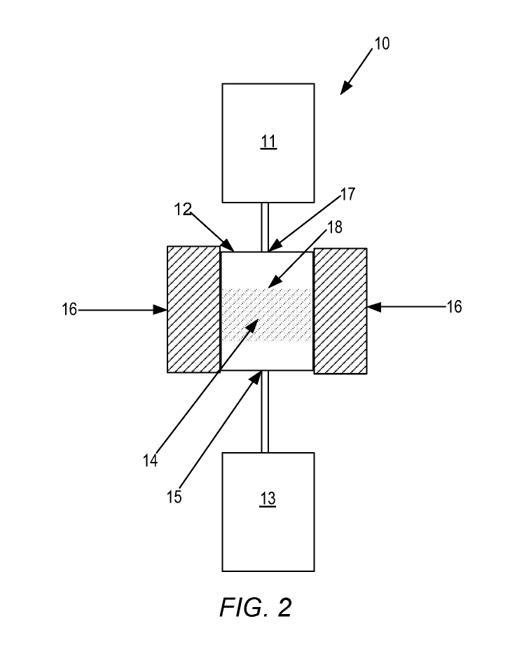

Referring to FIG. 2, a system 10 is illustrated, which can be used to convert alkyl halides to olefin hydrocarbon products with the nano-crystal and/or hierarchical SAPO-34 and MeAPSO-34 catalysts of the present invention. The system 10 can include an alkyl halide source 11, a reactor 12, and a collection device 13. The alkyl halide source 11 can be configured to be in fluid communication with the reactor 12 via an inlet 17 on the reactor. As explained above, the alkyl halide source can be configured such that it regulates the amount of alkyl halide feed entering the reactor 12. The reactor 12 can include a reaction zone 18 having the nano-platelet SAPO-34 catalyst 14 of the present invention. The amounts of the alkyl halide feed 11 and the catalyst 14 used can be modified as desired to achieve a given amount of product produced by the system 10. Non-limiting examples of reactors that can be used include fixed-bed reactors, fluidized bed reactors, bubbling bed reactors, slurry reactors, rotating kiln reactors, or any combinations thereof when two or more reactors are used. Fluid catalytic cracking (FCC)-type reactors or circulating catalyst bed reactors permit the regeneration of used/deactivated catalysts in a continuous process. The reactor 12 can include an outlet 15 for products produced in the reaction zone 18. The products produced can include ethylene, propylene and butylene. The collection device 13 can be in fluid communication with the reactor 12 via the outlet 15. Both the inlet 17 and the outlet 15 can be open and closed as desired. The collection device 13 can be configured to store, further process, or transfer desired reaction products (e.g., C₂-C₄ olefins) for other uses. By way of example only, FIG. 1 provides non-limiting uses of propylene produced from the catalysts and processes of the present invention. Still further, the system 10 can also include a heating source 16. The heating source 16 can be configured to heat the reaction zone 18 to a temperature sufficient (e.g., 325 to 375° C.) to convert the alkyl halides in the alkyl halide feed to olefin hydrocarbon products. Non-limiting examples of heating source 16 can be a temperature controlled furnace, heaters, heat exchangers and the like. Additionally, any unreacted alkyl halide can be recycled and included in the alkyl halide feed to further maximize the overall conversion of alkyl halide to olefin products. Further, certain products or byproducts such as butylene, C₅₊ olefins and C₂₊ alkanes can be separated and used in other processes to produce commercially valuable chemicals (e.g., propylene). This increases the efficiency and commercial value of the alkyl halide conversion process of the present invention.

2. Catalyst Activity/Selectivity

Catalytic activity as measured by alkyl halide conversion can be expressed as the % moles of the alkyl halide converted with respect to the moles of alkyl halide fed. In particular aspects, the combined selectivity of ethylene and propylene is at least 70%, preferably at least 80%, more preferably at least 90%, or most preferably 90% to 98% under certain reaction conditions, wherein the maximum combined space time yield (STY) of ethylene and propylene is at least 1/hr or 1/hr to 3/hr, and/or wherein the maximum conversion of alkyl halide is at least 65% or 70% to 80%. In certain instances, the selectivity of ethylene is about 40% or higher and the selectivity of propylene is about 30% or higher, wherein the maximum selectivity of ethylene is about 50% to 60% and the maximum selectivity of propylene is about 35% to 45%.

As an example, chloromethane (CH₃Cl) is used here to define conversion and maximum selectivity of products by the following equations (I) and (II):

$$\% {CH}_{3} Cl Conversion = \frac{( {CH}_{3} Cl ) ° - ( {CH}_{3} Cl )}{( {CH}_{3} Cl ) °} \times 100 , \qquad ( I )$$

where, (CH₃Cl)° and (CH₃Cl) are moles of methyl chloride in the feed and reaction product, respectively.

Maximum selectivity is defined as C-mole % and are defined for ethylene, propylene, and so on as follows:

$$\% Ethylene Selectivity = \frac{2 ( {C}_{2} {H}_{4} )}{( {CH}_{3} Cl ) ° - ( {CH}_{3} Cl )} \times 100 , \qquad ( II )$$

where the numerator is the carbon adjusted mole of ethylene and the denominator is the moles of carbon converted.

Maximum selectivity for propylene may be expressed as:

$$\% Propylene Selectivity = \frac{3 ( {C}_{3} {H}_{6} )}{( {CH}_{3} Cl ) ° - ( {CH}_{3} Cl )} \times 100 , \qquad ( III )$$

where the numerator is the carbon adjusted mole of propylene and the denominator is the moles of carbon converted.

### D. Materials

1. SAPO and MeAPSO Materials

SAPO, MeAPSO, hierarchical SAPO, and hierarchical MeAPSO catalysts are made from silicon (Si), aluminum (Al), and phosphorous (P) in various molar ratios. Non-limiting examples of silicon sources include colloidal silica, fumed silica, tetramethyl orthosilicate, tetraethyl orthosilicate, or tetraisopropyl orthosilicate. Non-limiting examples of aluminum sources include aluminum methoxide, aluminum ethoxide, aluminum isopropoxide, or aluminums tert-butoxide. Non-limiting examples of phosphorus sources include phosphoric acid. These compounds can be obtained from various commercial sources, of which Sigma Aldrich® (U.S.A) is a non-limiting example. Templating agents can be used to direct crystal growth, pore size and the like. Non-limiting examples of templating agents include amines, quaternary ammonium salts, or both. Non-limiting examples of amines include diethylamine, triethylamine, or morpholine. Non-limiting examples of quaternary ammonium salts include tetraethylammonium hydroxide (TEAOH), tetramethylammonium hydroxide (TMEOH), and tetrapropylammioum hydroxide (TPAOH). Templating agents are available from commercial sources, for example, Sigma Aldrich® (U.S.A).

The metal or metal oxide denoted “Me” in the MeASPO materials can include metals or metal oxides from Columns 7 to 12 of the Periodic Table or combinations thereof. Non-limiting examples of metals include manganese (Mn), magnesium (Mg), copper (Cu), cobalt (Co), iron (Fe), nickel (Ni), germanium (Ge), or zinc (Zn). The metals or metal oxides can be purchased from commercial manufactures such as Sigma-Aldrich®.

Mesopore-forming agents and crystal growth modifiers can be employed in the current methods include compounds that can assist in the assembly of the precursor materials and/or nanocrystals. In some embodiments, the mesopore-formation agent can be removed during calcination of the SAPO materials. Non-limiting examples of crystal growth modifiers include polyethylene glycol (PEG), hexadecyltrimethylammonium bromide (HDAB), cetyltrimethylammonium bromide (CTAB), poly(diallyldimethylammonium) (PDDAMA) salts, poly(diallydimethylammonium chloride (PDDAC), copolymers of ethylene oxide and propylene oxide (e.g., Pluronic® copolymers, BASF, USA), polyethylene oxide (PEO), polypropylene oxide (PPO), and polyimines such as polyethyleneimine (PEI). The polyethylene glycol (PEG) can be PEG 200, PEG 300, PEG 400, PEG 540, PEG 600, PEG 800, PEG 900, PEG 1000, PEG 1450, PEG 1540, PEG 2000, PEG 3000, PEG 4000, or PEG 6000. In a particular instance, PEG 300 is used. PEGs can be obtained from Spectrum® Chemical Mfg. Corp. (USA). Non-limiting examples of mesopore forming agents include the crystal growth modifiers listed above and carbon nanotubes or multi-wall carbon nanotubes.

2. Alkyl Halide Feed

The alkyl halide feed includes one or more alkyl halides. The alkyl halide feed may contain alkyl mono halides, alkyl dihalides, alkyl trihalides, preferably alkyl mono halide with less than 10% of other halides relative to the total halides. The alkyl halide feed may also contain nitrogen, helium, steam, and so on as inert compounds. The alkyl halide in the feed may have the following structure: CₙH₍₂ₙ₊₂₎₋ₘXₘ, where n and m are integers, n ranges from 1 to 5, preferably 1 to 3, even more preferably 1, m ranges 1 to 3, preferably 1, X is Br, F, I, or Cl. In particular aspects, the feed may include about 10, 15, 20, 40, 50 mole % or more of the alkyl halide. In particular embodiments, the feed contains up to 10 mole % or more of a methyl halide. In preferred aspects, the methyl halide is methyl chloride, methyl bromide, methyl fluoride, or methyl iodide, or any combination thereof. The feed stream can also include alcohol. In a particular embodiment, the feed stream includes less than 5 wt. % alcohol, preferably less than 1 wt. % alcohol, or preferably is alcohol free, and in one instance that alcohol is methanol.

The production of alkyl halide, particularly of methyl chloride (CH₃Cl) is commercially produced by thermal chlorination of methane at 400° C. to 450° C. and at a raised pressure. Catalytic oxychlorination of methane to methyl chloride is also known. In addition, methyl chloride is industrially made by reaction of methanol and HCl at 180° C. to 200° C. using a catalyst. Alternatively, methyl halides are commercially available from a wide range of sources (e.g., Praxair, Danbury, Conn.; Sigma-Aldrich Co. LLC, St. Louis, Mo.; BOC Sciences USA, Shirley, N.Y.). In preferred aspects, methyl chloride and methyl bromide can be used alone or in combination.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results. The materials used in the following examples are described in Table 1, and were used as-described unless specifically stated otherwise.

TABLE 1

| Material | Source |
|---|---|
| Colloidal Silica (Ludox AS-40), 40 wt. % | Sigma-Aldrich ® (USA) |
| SiO₂ | |
| Aluminum iso-propoxide | Sigma-Aldrich ® |
| (Al(O—i-Pr)₃), ≥98% purity | |
| Phosphoric acid (H₃PO₄) 85 wt. % aqueous | Sigma Aldrich ® |
| Hydrochloric acid (HCl), 37 wt. % HCl | Sigma-Aldrich ® |
| aqueous | |
| Tetraethylammonium hydroxide | SACHEM, Inc. (USA) |
| ((C₂H₅)₄N(OH)), 35 wt. % aqueous | Zeogen ™ |
| Water (deionized) | SABIC labs |
| PEG-300, P0108 | Spectrum (USA) |
| Cetyltrimethylammonium bromide (CTAB) | Sigma Aldrich ® |
| Manganese Acetate | Sigma Aldrich ® |
| SAPO-34 (Comparative Catalyst 1) | ACS Material ® |

### Example 1

### Water and Water Vapor/Steam Assisted of Dried Material Syntheses

Zeolite catalysts were prepared by the methods of the present invention. The inorganic reagents, organic template/structure directing agent, and solvent (water) listed in Table 1 were combined to form a homogeneous mixture/gel. When supersaturation of the inorganic reagents was reached, zeolite/SAPO nuclei begin to condense in the solution. After aging, with agitation, the gel for a period of time 24 hrs, the gel was dried at 90° C. until there was no noticeable water present in the material and the organic template was evaporated from the solution. The dried material was a powder.

Water Assisted Method:

The resulting dried material was then loaded into a polytetrafluoroethylene-lined autoclave along with a small portion of water which serves to redisperse the gel into solution.

Water Vapor/Steam Assisted Method:

The resulting dried material was placed in a small container that was placed into a larger container. A given amount of water was then placed in the larger container (without the dried and water coming into contact). Both containers were then placed into an autoclave at a specific temperature for a given amount of time.

After crystallization the catalysts were further calcined at 500° C. to 600° C. for 3 to 10 hours to afford the catalyst product. Table 2 lists the catalyst name, synthesis method and molar composition of the catalysts of the present invention.

TABLE 2

| No. | Catalyst Name | Synthesis Method | Molar Composition |
|---|---|---|---|
| 1a | SAPO-34 | Water | 2TEAOH:0.4SiO₂:1Al₂O₃:1P₂O₅:60H₂O |
| 1b | SAPO-34 | Vapor/Steam | 2TEAOH:0.4SiO₂:1Al₂O₃:1P₂O₅:60H₂O |
| 2a | MnAPSO-34 | Water | 2TEAOH:0.4SiO₂:1Al₂O₃:1P₂O₅:60H₂O:0.5MnO |
| 2b | MnAPSO-34 | Vapor/Steam | 2TEAOH:0.4SiO₂:1Al₂O₃:1P₂O₅:60H₂O:0.5MnO |

### Example 2

### Hierarchical SAPO-34 AND MnAPSO-34 Synthesis

Hierarchical SAPO catalysts were prepared according the methods of the present invention. The inorganic reagents, organic template/structure directing agent, crystal growth agent/mesopore-forming agent, and solvent (water) listed in Table 1 were combined to form a homogeneous mixture/gel that was processed in the manner described for Example 1 except that a mesopore-forming agent was added to the mixture and the mixture was dried. The remaining dried material can then be subjected to crystallization condition of either the water or steam assisted method as described in Example 1. Table 3 lists the hierarchical catalyst name, synthesis method and molar composition of the hierarchical catalysts of the present invention.

TABLE 3

| | Hierarchical | | |
|---|---|---|---|
| No. | Catalyst Name | Synthesis Method | Molar Composition |
| 3a | SAPO-34 | Water | 2TEAOH:0.4SiO₂:1Al₂O₃:1P₂O₅:60H₂O:0.01 CTAB |
| 3b | SAPO-34 | Vapor/Steam | 2TEAOH:0.4SiO₂:1Al₂O₃:1P₂O₅:60H₂O:0.01CTAB |
| 4a | SAPO-34 | Water | 2TEAOH:0.4SiO₂:1Al₂O₃:1P₂O₅:60H₂O:0.01PEG |
| 4b | SAPO-34 | Vapor/Steam | 2TEAOH:0.4SiO₂:1Al₂O₃:1P₂O₅:60H₂O:0.01PEG |
| 5a | MnAPSO-34 | Water | 2TEAOH:0.4SiO₂:1Al₂O₃:1P₂O₅:60H₂O:0.01CTAB:0.05MnO |
| 5b | MnAPSO-34 | Vapor/Steam | 2TEAOH:0.4SiO₂:1Al₂O₃:1P₂O₅:60H₂O:0.01CTAB:0.05MnO |
| 6a | MnAPSO-34 | Water | 2TEAOH:0.4SiO₂:1Al₂O₃:1P₂O₅:60H₂O:0.01PEG:0.05MnO |
| 6b | MnAPSO-34 | Vapor/Steam | 2TEAOH:0.4SiO₂:1Al₂O₃:1P₂O₅:60H₂O:0.01PEG:0.05MnO |

### Example 3

(Synthesis of Comparative Hierarchical MnAPSO-34 having Molar Composition aTEAOH:bSiO₂:cAl₂O₃:dP₂O₅:eH₂O:fX:gMnO, where X is the Mesopore-Forming Agent)

A first competitive hierarchical MeAPSO-34 catalyst (Comparative Catalyst 3) having molar composition of: 2TEAOH:0.4SiO₂:1Al₂O₃:1P₂O₅:60H₂O:0.01CTAB:0.05MnO was prepared by placing the synthesis mixture in an autoclave and heating to 200° C. for 24 hours.

### Example 4

### SAPO-34 Catalyst Characterization

Scanning Electron Microscopy (SEM) Analysis.

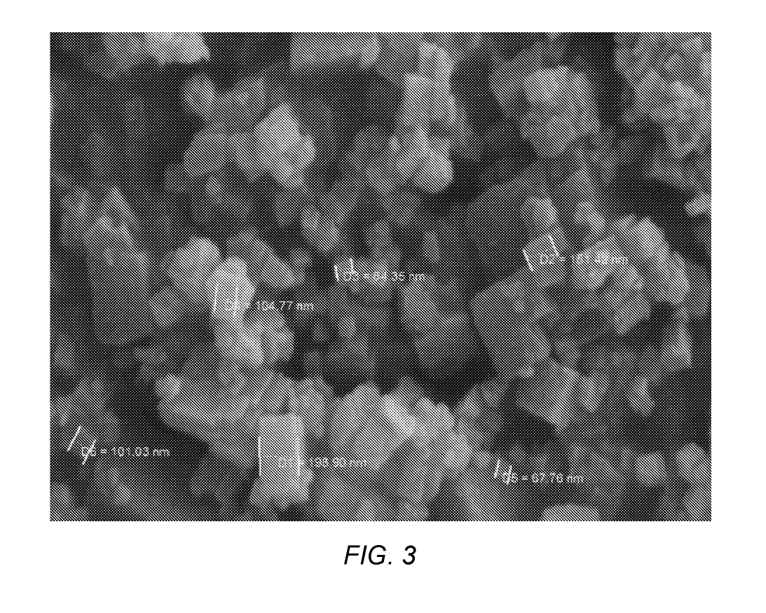

SEM analysis was performed on the inventive sample from Example 2 using a JSM-7800F-PRIME scanning electron microscope operating at 7 kV. FIG. 3 is a SEM image of the catalyst of Example 5a at magnification of 82.2 kx, viewing field of 3.37 micrometers. From the SEM, it was determined that regular cubed shaped nanoparticles of MnAPSO-34 with an average dimension of about 100 nm were formed.

### Example 5

### Production of Olefins from Chloromethane Using SAPO-34 Catalysts

General Procedure.

Catalysts of the present invention and comparative catalysts were tested for methyl chloride conversion by using a fixed-bed tubular reactor at about 450° C. for a period of 5 h. For catalytic testing the powder catalysts were pressed and then crushed and sized between 20 and 40 mesh screens. In each test a fresh load of sized (20-40 mesh) catalyst (1.0 g) was loaded in a stainless steel tubular (½-inch outer diameter) reactor. The catalyst was dried at 200° C. under N₂ flow (100 cm³/min) for an hour and then temperature was raised to 450° C. at which time N₂ was replaced by methyl chloride feed (100 cm³/min) containing 20 mol % CH₃Cl in N₂. The weight hourly space velocity (WHSV) of CH₃Cl was about 0.8 h⁻¹ to 3.0 h⁻¹ and reactor inlet pressure was about 0 MPa.

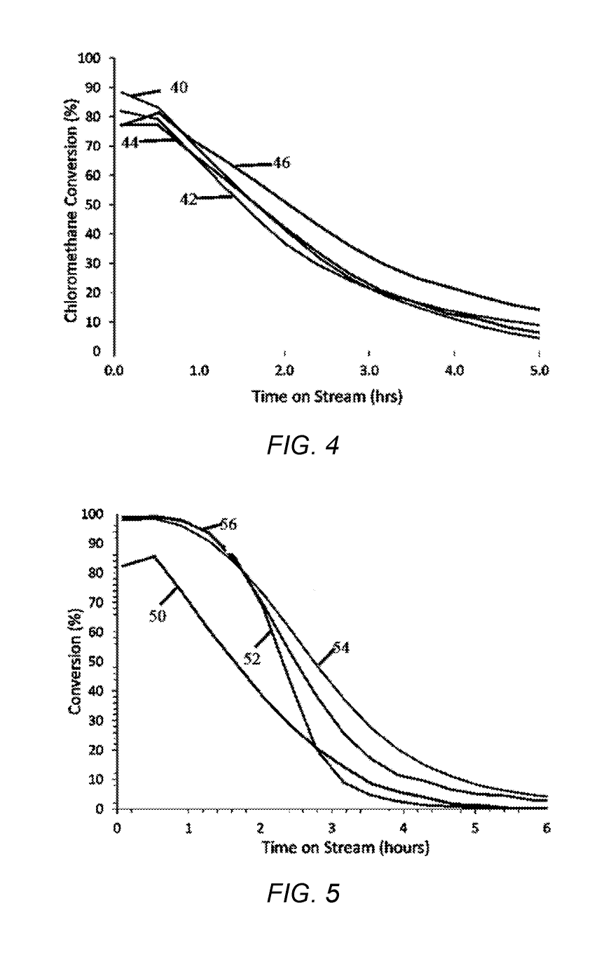

FIG. 4 shows the chloromethane conversion at versus time on stream for SAPO-34 catalysts made using the methods in Examples 1a, 3a and 7. In FIG. 4, data line 40 is a Comparative SAPO-34 catalyst 1, data line 42 is a SAPO-34 catalyst made using the methods of the present invention (Example 1a), data line 44 is comparative SAPO-34 catalyst 3, and data line 46 is a hierarchical SAPO-34 catalyst made with CTAB from Example 3a.

From the data in FIG. 4, it was determined that catalyst from Example 3a (data line 46) had a slower deactivation rate as compared to the other samples. Without wishing to be bound by theory, it is believed that a larger fraction of the mesopores present in SAPO-34 extend to the surface of the zeolite crystals, as compared to the other samples. Since the methods of the present invention provide very small crystals, it is believed that the incorporation of CTAB into a dry gel synthesis increased the likelihood that a greater portion of CTAB extended to the external surface of the SAPO-34 crystals, thereby changing the zeolite crystal size, which results in the increased productivity of light olefins.

### Example 6

### Production of Olefins from Chloromethane Using MnAPSO-34 Catalysts

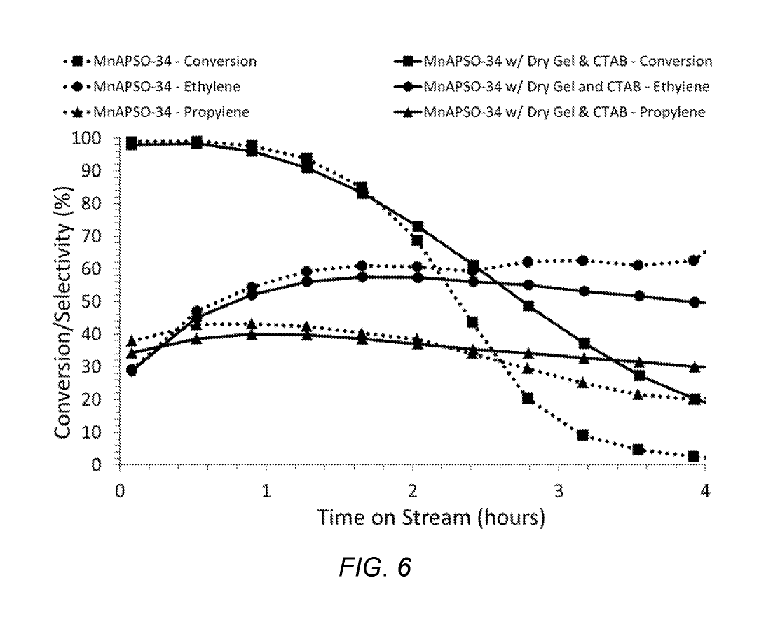

FIG. 5 shows chloromethane conversion at a temperature of 450° C., a weighted hourly space velocity of 3 hr⁻¹ and a pressure of 0 psig versus time on stream for MnAPSO-34 catalysts of Examples 2a and 5a of the present invention, comparative catalyst 2, and comparative catalyst 1. In FIG. 5 data line 50 and data line 52 are comparative SAPO-34 catalyst 1 and comparative MnAPSO-34 catalyst 2, data line 54 is the nano-hierarchical MnAPSO-34 catalyst 5a of the present invention, and data line 56 is nano-MnAPSO-34 catalyst 2a of the present invention. FIG. 6 shows, the selectivity data for ethylene and propylene for the MnAPSO-34 catalyst (catalyst 5A) of the present invention and the comparative catalyst (comparative catalyst 2).

From the data in FIGS. 5 and 6, the catalytic activity of all the MnAPSO-34 catalysts exceeds that of conventionally prepared SAPO-34 (comparative catalyst 1) and the conventionally prepared MnAPSO-34. For the MnAPSO catalysts, Catalyst 5a, data line 54, showed the slowest deactivation rate. Without wishing to be bound by theory, it is believed that a larger fraction of the mesopores present in nano-hierarchical catalyst 5a (MnAPSO-34) of the present invention allow better diffusion of reactants and products through the catalyst. Since the methods of the present invention provide very small crystals (See, FIG. 3), it is believed that the incorporation of CTAB into a dry gel synthesis increased the amount of mesopores and inhibited agglomeration of crystals during the crystallization step, thereby changing the zeolite crystal size, which results in the increased productivity of light olefins.

1. A method for preparing a silicoaluminophosphate (SAPO)-34 molecular sieve, the method comprising:
(a) obtaining an aqueous mixture comprising water, a silicon source, an aluminum source, a phosphorous source, and a templating agent;
(b) drying the mixture to obtain a dried material comprising SAPO-34 precursor material loaded with the templating agent;
(c) contacting the dried material with water and subjecting the material to crystallization conditions to obtain a SAPO-34 crystalline material loaded with the templating agent; and
(d) removing the templating agent from the crystalline material to obtain the SAPO-34 molecular sieve.

2. The method of claim 1, wherein step (c) comprises suspending the dried material in an aqueous solution to form a suspension and subjecting the suspension to a temperature of 180° C. to 210° C. for 12 hours to 36 hours under autogenous pressure to obtain the SAPO-34 crystalline material loaded with the templating agent.

3. The method of claim 1, wherein step (c) comprises contacting the dried material with water vapor or steam and subjecting the material to a temperature of 180° C. to 210° C. for 12 hours to 36 hours under autogenous pressure to obtain the SAPO-34 crystalline material loaded with the templating agent.

4. The method of claim 1, wherein the obtained SAPO-34 molecular sieve has a microporous structure and is in particulate form having an average particle size of 50 nm to 500 nm or 50 nm to 200 nm.

5. The method of claim 1, wherein the aqueous mixture in step (a) has a molar composition of:
aR:bSiO₂:cAl₂O₃:dP₂O₅:eH₂O
where R is the templating agent, and 0<a≤4, 0<b≤1, 0<c≤1, 0<d≤1, and e is 30 to 80.

6. The method of claim 1, wherein:
the aqueous mixture in step (a) further comprises a crystal growth modifier, a mesopore-forming agent or both;
the dried material from step (b) and the crystalline material from step (c) are each loaded with the templating agent and the crystal growth modifier; and
the templating agent and the crystal growth modifier are each removed from the crystalline material to obtain the SAPO-34 molecular sieve.

7. The method of claim 6, wherein the obtained SAPO-34 molecular sieve is in particulate form having an average particle size of 50 nm to 500 nm or 50 nm to 200 nm and/or hierarchical structure of micropores and mesopores.

8. The method of claim 6, wherein the aqueous mixture in step (a) has a molar composition of:
aR:bSiO₂:cAl₂O₃:dP₂O₅:eH₂O:fX,
where R is the templating agent and X is the crystal growth modifier, mesopore forming agent or both, and a is 0<a≤4, b is 0<b≤1, c is 0<c≤1, d is 0<d≤1, e is 30 to 80, and f is 0<f≤1.

9. The method of claim 6, wherein the crystal growth modifier is a polyethylene glycol (PEG), cetyltrimethylammonium bromide (CTAB), polyimine, polyethyleneimine (PEI), or any combination thereof.

10. The method of claim 6, wherein the crystal growth modifier forms mesopores upon calcination.

11. The method of claim 7, wherein the mesopore-forming agent is a carbon nanotube.

12. The method of claim 1, wherein the aqueous mixture further comprises a metal (Me) source, wherein Me is manganese, magnesium, copper, cobalt, iron, nickel, germanium, or zinc or an oxide thereof.

13. The method of claim 12, wherein the aqueous mixture in step (a) has a molar composition of:
aR:bSiO₂:cAl₂O₃:dP₂O₅:eH₂O:gMe_{y}O_{z},
where R is the templating agent, 0<a≤4, 0<b≤1, 0<c≤1, 0<d≤1, e is 30 to 80, and 0<g≤1, y is 1 to 2, and z is 1 to 3.

14. The method of claim 13, wherein the aqueous mixture in step (a) has a molar composition of:
aR:bSiO₂:cAl₂O₃:dP₂O₅:eH₂O:fX:gMe_{y}O_{z},
where R is the templating agent, X is the crystal growth modifier, the mesopore-forming agent or both, 0<a≤4, 0<b≤1, 0<c≤1, 0<d≤1, e is 30 to 80, 0<f≤1, and 0<g≤1, y is 1 to 2, and z is 1 to 3.

15. The method of claim 1, wherein the templating agent is an amine, a quaternary ammonium salt or both.

16. The method of claim 1, wherein the drying step (b) removes substantially all of the templating agent from the aqueous mixture other than the templating agent that is loaded into the SAPO-34 precursor material.

17. The method of claim 1, wherein:
the drying step (b) includes subjecting the mixture to a temperature of 80° C. to 110° C.; and/or
the removing step (d) comprises subjecting the crystalline material to a temperature of 500° C. to 600° C. for 3 hours to 10 hours.

18. A silicoaluminophosphate (SAPO)-34 molecular sieve having an average particle size of 50 nm to 500 nm.

19. The silicoaluminophosphate (SAPO)-34 molecular sieve of claim 18, wherein the molecular sieve has a hierarchical structure of micropores and mesopores.

20. A method for converting an alkyl halide to an olefin, the method comprising contacting the silicoaluminophosphate (SAPO)-34 molecular sieve of claim 16 with a feed comprising an alkyl halide under reaction conditions sufficient to produce an olefin hydrocarbon product comprising C₂-C₄ olefins.
